(19) United States
(12) Patent Application Publication
(10) Pub. No.: US 2018/0177405 A1
(43) Pub. Date: Jun. 28, 2018

(54) OBJECT INFORMATION ACQUIRING APPARATUS AND OBJECT INFORMATION ACQUIRING METHOD

(71) Applicant: CANON KABUSHIKI KAISHA, Tokyo (JP)

(72) Inventors: Daisuke Sasaguri, Yokohama-shi (JP); Atsushi Takahashi, Ebina-shi (JP)

(21) Appl. No.: 15/580,112

(22) PCT Filed: Jul. 11, 2016
(86) PCT No.: PCT/JP2016/070924

§ 371 Date: Dec. 6, 2017

(30) Foreign Application Priority Data
Jul. 21, 2015 (JP) 2015-144047
May 30, 2016 (JP) 2016-107261

(51) Int. Cl. A61B 5/00; A61B 5/145; A61K 49/00

(52) U.S. Cl. CPC A61B 5/0095; A61B 5/145; A61K 49/0056; A61K 49/0034; A61K 49/0054; A61B 5/14542

(57) ABSTRACT

An object information acquiring apparatus includes: a light source radiating first light having a first wavelength λ1 and second light having a second wavelength λ2; a detection unit detecting an acoustic wave generated from an object irradiated with the light, and converting the acoustic wave into a detection signal; and a signal processing unit acquiring characteristic information of the inside of the object based on the detection signal. The signal processing unit acquires the characteristic information by performing subtraction processing between a signal generated as a result of the first light being absorbed by hemoglobin and a signal generated as a result of the second light being absorbed by hemoglobin.

### TECHNICAL FIELD

The present invention relates to an object information acquiring apparatus and an object information acquiring method.

### BACKGROUND ART

Aggressive research on optical imaging apparatuses, which radiate light from a light source such as a laser into a living body and image information on the inside of the living body that is acquired based on the entered light, is ongoing. Photoacoustic imaging (PAI) is an example of such optical imaging technology.

In photoacoustic imaging, pulsed light generated from a light source is radiated into a living body, an acoustic wave (typically an ultrasonic wave), generated from bio-tissue which has absorbed the energy of the pulsed light propagating in the living body, is detected, and bio-information is imaged based on the detected signal (PA signal). In other words, by utilizing the difference of the absorptivity of the light energy between a target segment, such as blood, and peripheral tissue thereof, an elastic wave, generated when the object segment absorbs the radiated light energy and expands instantaneously, is detected by a photoacoustic detector. By mathematically analyzing this detected signal, the optical characteristic distribution inside the living body, particularly the initial sound pressure distribution, the light energy absorption density distribution, the absorption coefficient distribution or the like, can be acquired.

In PAI, the initial sound pressure P₀ of the acoustic wave, which is generated from the light absorber in the object, is given by the following Expression (1).

P₀=Γ·μa·Φ (1)

Here Γ denotes a Grüneisen coefficient that is determined by dividing the product of a volume expansion coefficient β and a square of the sound velocity c by a specific heat at constant pressure Cp. It is known that if the object is determined, the value of Γ is approximately constant. μa denotes an absorption coefficient of a light absorber. Φ denotes a light quantity at a position of the light absorber (that is, the quantity of light radiated to the light absorber, also called “light fluence”). Since Γ does not depend on wavelength, it can be understood that the signal intensity depends on the product of the absorption coefficient μa and the light quantity Φ.

The initial sound pressure P₀, generated in the light absorber in the object, propagates through the object as an acoustic wave, and is detected by an acoustic wave detector disposed on the surface of the object. The temporal change in the detected sound pressure of the acoustic wave is measured, and such an image reconstruction method as a back-projection method is used for the measurement result, whereby the initial sound pressure distribution P₀ can be calculated. By dividing the calculated initial sound pressure distribution P₀ by the Grüneisen coefficient Γ, the distribution of the product of μa and Φ, in other words, the light energy density distribution, can be acquired. If the light quantity distribution Φ inside the object is known, the absorption coefficient distribution μa can be acquired by dividing the light energy density distribution by the light quantity distribution Φ.

Hemoglobin is an example of a light absorbing component inside a living body. Hemoglobin can be oxyhemoglobin or deoxyhemoglobin depending on the combination state of the oxygen, and each has a different light absorption characteristic.

A contrast agent is a light absorbing component that is administered into the object, and has a light absorption characteristic with respect to the light radiated into the object. If photoacoustic measurement is performed for an object after administering the contrast agent, a photoacoustic image corresponding to the distribution of the contrast agent is acquired. For example, if a contrast agent that is specifically drawn to a cancer is administered, then a position, characteristic and the like of the cancer can be detected.

In the case of performing PAI in the state after administering the contrast agent, the acquired signal includes both the signal originating in hemoglobin and the signal originating in the contrast agent, and therefore these signals must be separated in order to check the distribution of the contrast agent. Further, the light absorption characteristic of oxyhemoglobin is different from that of deoxyhemoglobin. In other words, components having three types of different light absorption characteristics coexist if the contrast agent is included. Generally if a light absorber having three different light absorption characteristics exists, the PA signals must be acquired at a minimum of three wavelengths. This results in increasing the measurement time.

Japanese Patent Application Laid-Open No. 2008-261784 (hereafter Patent Literature 1) attempts to solve this problem by a subtraction method. This is a method of subtracting two captured images. According to Patent Literature 1, an image before administering the contrast agent and an image after administering the contrast agent are acquired, and subtraction processing is performed for these images, whereby only the signal originating in the contrast agent can be acquired.

A PAI-based subtraction processing technique is disclosed in Japanese Patent Application Laid-Open No. 2013-055988 (hereafter Patent Literature 2). According to Patent Literature 2, in order to remove undesired artifacts generated on the surface of the object, PA signals are acquired at two different wavelengths, and subtraction processing is performed on these PA signals to extract only the signals generated inside the object.

Robert A. Kruger et al., “Thermoacoustic Molecular Imaging of Small Animals”, Molecular Imaging, Vol. 2, No. 2, April 2003, pp. 113-123 (Non Patent Literature 1) discloses a technique of acquiring PA signals at two different wavelengths and performing subtracting processing on the acquired PA signals, so as to extract signals generated from the contrast agent (indocyanine green) inside the object.

### CITATION LIST

### Patent Literature

Patent Literature 1: Japanese Patent Application Laid-Open No. 2008-261784
Patent Literature 2: Japanese Patent Application Laid-Open No. 2013-055988

### Non Patent Literature

Non Patent Literature 1: Robert A. Kruger et al., “Thermoacoustic Molecular Imaging of Small Animals”, Molecular Imaging Vol. 2, No. 2, April 2003, pp. 113-123

### SUMMARY OF INVENTION

### Technical Problem

In the case of Patent Literature 1, regions of which signals are different between before and after the administration of the contrast agent are extracted, hence signals originating in the contrast agent can be identified. However, an accurate positional alignment of the images is necessary to perform subtraction of images before and after the administration, and in practical terms, it is difficult to capture images exactly at a same position and in a same state. Especially when the interval between imaging before and after administration of the contrast agent is long and consecutive imaging cannot be performed, it is impossible to capture the images at exactly a same position. Further, performing image capturing at least twice (before and after administration) is required, which results in a longer measurement time.

The subtraction processing in PAI disclosed in Patent Literature 2 is an invention to extract a hemoglobin signal inside the object by erasing artifacts on the surface of the object, and is different from the imaging technique to remove the hemoglobin signal.

The Non Patent Literature 1 discloses the imaging conditions to erase the PA signal from hemoglobin and extract the PA signals of the contrast agent. However, the output characteristic of the light source and the intensity of the radiated light are not considered here, therefore it is insufficient as an advanced technique to erase the signal originating in hemoglobin and extract a signal originating in the contrast agent.

With the foregoing in view, it is an object of the present invention to reduce the influence of components other than the measurement target in the information acquired by PAI.

### Solution to Problem

The present invention provides an object information acquiring apparatus, comprising:

a light source configured to radiate first light having a first wavelength λ1 and second light having a second wavelength λ2;

a detection unit configured to detect an acoustic wave, which is generated from an object irradiated with the light from the light source, and convert the acoustic wave into a detection signal;

a signal processing unit configured to acquire characteristic information of an inside of the object based on the detection signal; and

a light intensity acquiring unit configured to acquire incident light intensity of the light that is radiated from the light source into the object, wherein

the signal processing unit acquires the characteristic information by performing subtraction processing between a signal generated as a result of the first light being absorbed by hemoglobin and a signal generated as a result of the second light being absorbed by hemoglobin,

the first wavelength is 780 nm or more and 810 nm or less, and the second wavelength is 840 nm or more and 920 nm or less, and

an adjustment is performed so that Φ(λ1)≤Φ(λ2) is satisfied, and a difference between the Φ(λ1) and Φ(λ2) is within a predetermined range, where the Φ(λ1) denotes the incident light intensity of the first light to the object, and the Φ(λ2) denotes the incident light intensity of the second light to the object, which are acquired by the light intensity acquiring unit.

The present invention also provides an object information acquiring method, comprising:

a step of radiating first light having a first wavelength λ1 and second light having a second wavelength λ2 from a light source;

a step of a detection unit detecting an acoustic wave, which is generated from an object irradiated with light from the light source, and converting the acoustic wave into a detection signal;

a step of a signal processing unit acquiring characteristic information of an inside of the object based on the detection signal;

a step of acquiring incident light intensity of the light that is radiated from the light source into the object; and

a step of performing an adjustment so that Φ(λ1) Φ(λ2) is satisfied, and a difference between the Φ(λ1) and Φ(λ2) is within a predetermined range, where Φ(λ1) denotes the incident light intensity of the first light to the object, and Φ(λ2) denotes the incident light intensity of the second light, wherein

the first wavelength is 780 nm or more and 810 nm or less, and the second wavelength is 840 nm or more and 920 nm or less, and

in the step of acquiring the characteristic information, the signal processing unit acquires the characteristic information by performing subtraction processing between a signal generated as a result of the first light being absorbed by hemoglobin and a signal generated as a result of the second light being absorbed by hemoglobin.

### Advantageous Effects of Invention

According to the present invention, the influence of components other than the measurement target in the information acquired by PAI can be reduced.

Further features of the present invention will become apparent from the following description of exemplary embodiments (with reference to the attached drawings).

### BRIEF DESCRIPTION OF DRAWINGS

FIG. 1 is a diagram depicting an object information acquiring apparatus;

FIG. 2 is a graph depicting the light absorption characteristics of hemoglobin;

FIG. 3 is a flow chart depicting a processing flow of a detection signal;

FIGS. 4A to 4C show example of a detection signal and a difference signal;

FIGS. 5A to 5C show example of images based on first data to third data;

FIG. 6 is a graph depicting the light absorption characteristics of hemoglobin and a contrast agent; and

FIG. 7 is a diagram depicting a detailed configuration of the object information acquiring apparatus.

### DESCRIPTION OF EMBODIMENTS

Preferred embodiments of the present invention will now be described with reference to the drawings. The dimensions, materials, shapes, relative positions and the like of the components described herein below should be appropriately changed depending on the configuration and various conditions of the apparatus to which the invention is applied. Therefore these component aspects are not intended to limit the scope of the invention.

This embodiment is related to a technique to detect an acoustic wave propagated from an object, and generate and acquire characteristic information of the inside of the object. Therefore this embodiment can be regarded as an object information acquiring apparatus or a control method thereof, or an object information acquiring method and a signal processing method. Furthermore, this embodiment can also be regarded as a program that causes an information processing apparatus constituted by hardware resources, such as a CPU and memory, to execute these methods, and a storage medium storing this program.

The object information acquiring apparatus of this embodiment includes an apparatus using a photoacoustic effect, configured to receive an acoustic wave generated inside an object by radiating light (electromagnetic wave) into the object, and acquire characteristic information of the object as image data. The characteristic information according to this embodiment is information on a characteristic value corresponding to each of a plurality of positions inside the object, which is generated using the received signals acquired by receiving the photoacoustic wave.

The characteristic information acquired by this embodiment is a value reflecting the absorptivity of light energy. For example, the characteristic information includes the generation source of an acoustic wave generated by the radiation of the light, the initial sound pressure inside the object, the light energy absorption density or absorption coefficient derived from the initial sound pressure, and the concentration of a substance constituting a tissue. Oxygen saturation distribution can be calculated by determining the oxyhemoglobin concentration and the deoxyhemoglobin concentration as the substance concentration. Glucose concentration, collagen concentration, melanin concentration, volume fraction of fat and water and the like can also be determined. Furthermore, two-dimensional or three-dimensional characteristic information distribution can be acquired based on the characteristic information at each position inside the object. Distribution data can be generated as image data.

If the photoacoustic measurement is performed in a state where a contrast agent has been administered into the object, the characteristic information distribution reflecting the light absorption characteristic of the contrast agent can be acquired.

The acoustic wave according to this embodiment is typically an ultrasonic wave, including an elastic wave called a “sound wave” and an “acoustic wave”. An electric signal converted from an acoustic wave using a probe or the like is also called an “acoustic signal”. However the terms for an ultrasonic wave or an acoustic wave used in this description are not intended to limit the wavelength of the elastic wave. An acoustic wave generated by the photoacoustic effect is called a “photoacoustic wave” or a “light-induced ultrasonic wave”. An electric signal originating in a photoacoustic wave is also called a “photoacoustic signal”.

The object information acquiring apparatus according to the following embodiment may be used for the diagnosis of vascular diseases of humans and animals, and for the follow up observation of chemotherapy, for example.

### Overview of Configuration of Embodiment

The object information acquiring apparatus of this embodiment is basically configured by a detection unit that detects an acoustic wave and outputs a detection signal, and a signal processing unit that acquires characteristic information of the object based on the detection signal. When the detection unit detects a photoacoustic wave that is generated by the radiation of the light from the light source into the object, into which a contrast agent has been administered, characteristic information originating in the contrast agent can be acquired.

Here the light source can radiate at least a first light (wavelength λ1) and a second light (wavelength λ2, which is different from λ1). The light quantity at distance d in the object is assumed to be Φ(wavelength, d). Then the light quantity on the surface of the object (depth 0) is expressed as Φ(λ1, 0) and Φ(λ2, 0). These expressions correspond to the irradiation light quantity Φ(λ1) and Φ(λ2) respectively. These irradiation light quantity values change to Φ(λ1, d) and Φ(λ2, d) at the position of which depth from the surface of the object is d (d≥0) as a result of the absorption and scattering inside the object. The light quantity on the surface of the object can also be called “incident light intensity”.

Here the light absorption coefficients of oxyhemoglobin at λ1 and λ2 are assumed to be μ_{HbO2}(λ1) and μ_{HbO2}(λ2) respectively. The light absorption coefficients of deoxyhemoglobin at λ1 and λ2 are assumed to be μ_{Hb}(λ1) and μ_{Hb}(λ2) respectively.

In this embodiment, the irradiation light quantity (Φ(λ1, 0), Φ(λ2, 0)) is adjusted such that each product of the light absorption coefficient and the light quantity satisfies Expression (2) and Expression (3) at depth d inside the object. The light quantity can be adjusted using, e.g., the estimation result based on the scattering coefficient and the absorption coefficient inside the object, and using a rough estimate based on the depth d.

μ_{HbO2}(λ1)×Φ(λ1,d)≤μ_{HbO2}(Δ2)×Φ(λ2,d) (2)

μ_{Hb}(λ1)×Φ(λ1,d)≤μ_{Hb}(λ2)×Φ(λ2,d) (3)

Here oxyhemoglobin and deoxyhemoglobin are collectively referred to as hemoglobin (H). Based on Γ (constant) and sound pressure P at each wavelength determined by Expression (1) mentioned above, a value of the “signal of hemoglobin (signal S_{H}(λ1), signal S_{H}(λ2))” is estimated. Then for the signal of hemoglobin, the above Expressions (2) and (3) can be expressed as the following Expression (2)′.

S_{H}(λ1)≤S_{H}(λ2) (2)′

The detection unit acquires an electric signal for each light irradiation at each wavelength, and saves the electric signal as data. The signal processing unit calculates the difference between the first data originating in the radiation of the first light and the second data originating in the radiation of the second light. Thereby the signals of hemoglobin inside the object are reduced or removed.

Under these measurement conditions, the light absorption coefficients of the contrast agent at wavelength λ1 and wavelength λ2 are assumed to be μ_{CA}(λ1) and μ_{CA}(λ2) respectively. Then only the signal originating in the contrast agent can be extracted if each product of the light absorption coefficient and the light quantity satisfies the following Expression (4).

μ_{CA}(λ1)×Φ(λ1,d)>μ_{CA}(λ2)×Φ(λ2,d) (4)

### EMBODIMENT

### (Basic Configuration)

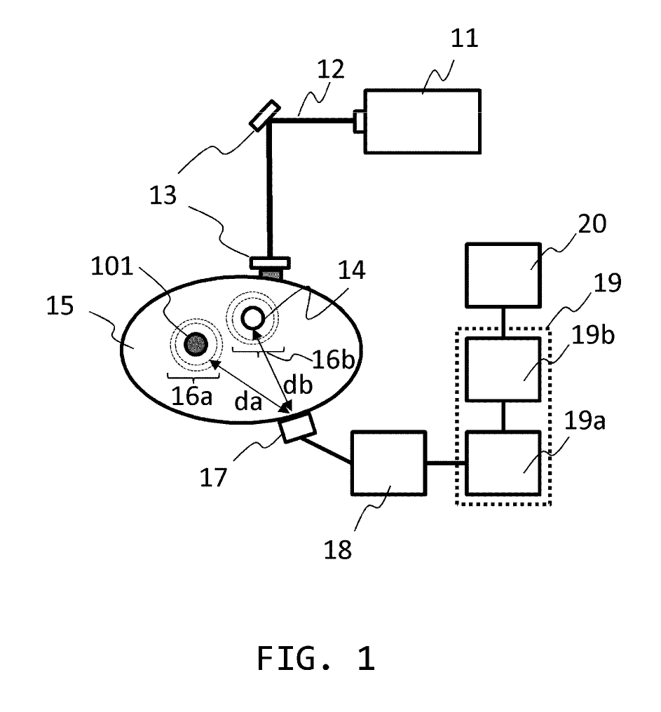

The configuration of the object information acquiring apparatus will be described with reference to FIG. 1. The apparatus has a basic hardware configuration of a light source 11, an acoustic wave detector 17, a signal processor 19 and a light intensity acquiring unit (not illustrated).

A pulsed light 12 emitted from the light source 11 is guided, while being processed into a desired light distribution shape by an optical system 12, and is radiated into an object 15. A light absorber 101 that absorbs light originating in the contrast agent (segments where the contrast agent administered into the object exists) and a light absorber 14 that absorbs light originating in the hemoglobin (e.g. blood vessels), exist inside the object 15. When a portion of energy of the light that was propagated after irradiation is absorbed by these light absorbers, an acoustic wave 16 is generated respectively by thermal expansion. The acoustic wave detector 17 detects the acoustic wave 16 and outputs it as a detection signal. A signal collector 18 performs such processing as amplification and digital conversion on the detection signal. The signal processor 19 performs predetermined processing on the signal and generates characteristic information (e.g. image data) of the object. A display device 20 displays the image data. In this embodiment, the acoustic wave detector corresponds to the detection unit, and the signal processor corresponds to the signal processing unit.

### (Wavelength Selection Method)

In this embodiment, the first light (having first wavelength λ1) and the second light (having second wavelength λ2), of which wavelengths are different from each other, are used as the light from the light source. If it is assumed that the light quantity of λ1 and that of λ2 are the same at depth d in the object, to simplify description, then Expression (2) and Expression (3) are established by selecting wavelength ranges in which Expression (5) and Expression (6) are established simultaneously. Therefore if the signal originating in the second light (an image captured with the second light) is subtracted from the signal originating in the first light (an image captured with the first light) under these conditions, the signals of hemoglobin, whether it is hemoglobin or deoxyhemoglobin, can be erased. If the signal intensity becomes a minus value as a result of the subtraction, the signal intensity can be set to 0.

μ_{HbO2}(λ1)≤μ_{HbO2}(λ2) (5)

μ_{Hb}(λ1)≤μ_{Hb}(λ2) (6)

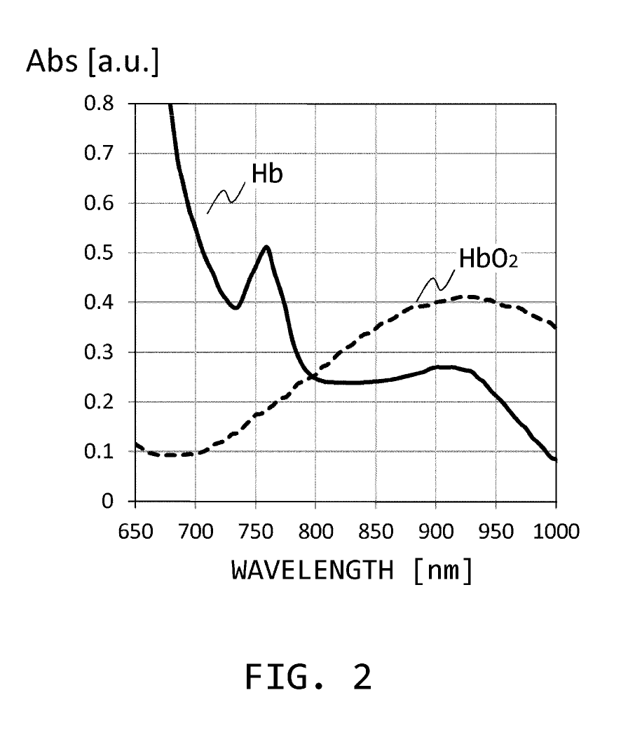

Now the first light and the second light will be described in detail. FIG. 2 shows the light absorption characteristic of hemoglobin. The abscissa of FIG. 2 indicates the wavelength, and the ordinate of FIG. 2 indicates the degree of absorption. As FIG. 2 shows, oxyhemoglobin (Hb) and deoxyhemoglobin (HbO₂) present different light absorption characteristics. In a living body, the existing ratio between oxyhemoglobin and deoxyhemoglobin differs depending on the measurement segment. For example, the ratio of deoxyhemoglobin is high in a vein. And in neo-vessels that exist around a tumor, the ratio of oxyhemoglobin is high since the arterial blood amount is high. The signal acquired in PAI has a signal characteristic and intensity according to the absorption characteristic of hemoglobin, depending on the wavelength of light that is radiated.

Here the focus is on a wavelength range from 780 nm to 920 nm. In this wavelength range, the light absorption characteristic of deoxyhemoglobin tends to be constant or slightly increases as the wavelength increases, and the light absorption characteristic of oxyhemoglobin tends to increase as the wavelength increases. Therefore Expression (5) and Expression (6) can be satisfied if the first wavelength λ1 and the second wavelength λ2 are selected in this wavelength range under the condition λ1<λ2.

In this wavelength range, the first wavelength λ1 and the second wavelength λ2 are selected, and the light having the selected wavelength λ1 or λ2 is radiated into the object. In this case, regardless of the combined ratio of hemoglobin and oxygen, the signal intensity generated from blood vessels that absorbed the second light is equivalent to or relatively higher than the signal intensity generated from the blood vessels that absorbed the first light. Therefore the signal of hemoglobin, determined by subtracting the detection signal originating in the second light from the detection signal originating in the first light, becomes 0 or less. As a result, a captured image, where the influence of the signal originating in hemoglobin is erased from the first light, can be acquired. If the subtraction result is a minus value, the value can be replaced with 0.

(Enhancing Signal Originating in Contrast Agent)

If a contrast agent exists inside the object, and two wavelengths of light, having similar light absorption coefficients by the contrast agent, are selected, the difference between the acquired signals originating in the contrast agent also becomes small. This phenomena could occur when the wavelengths λ1 and λ2 are relatively close. As a result, the signal intensity of the contrast agent acquired by the subtraction processing becomes small, and the signal may not be identified at all. Therefore if the contrast agent is used, it is preferable to select wavelengths that satisfy Expression (5) and Expression (6), and of which the light absorption difference by the contrast agent is as large as possible.

For example, in the case of a contrast agent combined with such a polymer as ICG or ICG-PEG, the first wavelength λ1 to be selected is preferably a wavelength with which the light absorption coefficient μ_{CA}(λ1) of the contrast agent is as high as possible in a wavelength range that satisfies Expression (5) and Expression (6). This wavelength range is preferably 780 nm to 810 nm. The second wavelength λ2 to be selected is preferably a wavelength with which the light absorption coefficient μ_{CA}(λ2) is as low as possible in a wavelength range that satisfies Expression (5) and Expression (6). This wavelength range is preferably 840 nm to 920 nm.

For example, it is assumed that 797 nm is selected for the first wavelength λ1, and 840 nm is selected for the second wavelength λ2. In this case, μ_{CA}(λ1) becomes about 10 times μ_{CA}(λ2). Therefore even if the subtraction processing is performed on the signals imaged with these two wavelengths, this signal of the contrast agent can be detected at a 90% or higher intensity of the signal of the contrast agent imaged with the first wavelength, hence the signal of the contrast agent can be acquired with certainty. If 797 nm is selected for the first wavelength λ1 and 850 nm is selected for the second wavelength λ2, μ_{CA}(λ1) becomes about 20 times μ_{CA}(λ2). Therefore even if the subtraction processing is performed on the signals imaged with these two wavelengths, the signals of the contrast agent can be detected at 95% or higher intensity compared with the signal of the contrast agent imaged with the first wavelength, therefore the signal of the contrast agent can be identified with certainty.

(Adjusting Irradiation Light Quantity: Removing Hemoglobin Signal)

Adjusting the irradiation light quantity of the first light and the second light to be radiated into the object will be described next. To erase the signal of hemoglobin which exists at depth d in the object, Expression (2) and Expression (3) must be established. If the wavelengths are selected according to the above mentioned method, Expression (5) and Expression (6) are established. Therefore to establish Expression (2) and Expression (3), the relationship with Expression (7) should be established for the light quantity at depth d from the surface of the object.

Φ(λ1,d)≤Φ(λ2,d) (7)

To examine the light quantity inside the object, the influence of scattering, attenuation, absorption and the like after irradiation must be considered. Here it is assumed that the irradiation light quantity of the light having wavelength λ, at a timing when the light is radiated from the light source into the object, is Φ(λ, 0). It is also assumed that light is radiated into a region that is wider than the thickness of the object, and the light propagates inside the object as a plane wave. In this case, the light quantity distribution Φ is given by the following Expression (8).

Φ(λ,d)=Φ(λ,0)·exp(−μ_{eff}(λ)·d) (8)

Here μ_{eff}(λ) denotes an average effective attenuation coefficient of the object at wavelength λ. Φ(λ, 0) denotes the light quantity (irradiation light quantity) that entered from the light source into the object. The depth d is a distance from the region on the surface of the object, to which light is radiated from the light source (light irradiation region), to the light absorber inside the object, that is, the depth d is the depth of the light absorber.

An absorption coefficient (μₐ), an equivalent scattering coefficient (μₛ′), and an effective attenuation coefficient (μ_{eff}), which are optical coefficients in the living body, have the relationship given by the following Expression (9).

μ_{eff}(λ)=√(3μₐ(λ)×(μₛ′(λ)+μₐ(λ))) (9)

In the wavelength range 450 nm to 950 nm, it is known that μₐ(λ1)≥μₐ (λ2) and μₛ′(λ1)>μₛ′(λ2) are established if the selected first wavelength λ1 and second wavelength λ2 have a relationship of λ1<λ2. Therefore according to Expression (9), the effective attenuation coefficient μ_{eff}(λ) always has the relationship μ_{eff}(λ1)>μ_{eff}(λ2), and it is known that λ1 has a greater attenuation of light quantity inside the object than λ2.

As a result, if the light source is adjusted such that the light having wavelength λ1 and light having wavelength λ2, selected as described above, have the same light quantity at depth 0 inside the object, then at depth d in the object, the quantity of light having the first wavelength λ1 always becomes lower than the quantity of light having the second wavelength λ2, as shown in Expression (8) and Expression (9), and satisfies Expression (7). As a consequence, the signals of hemoglobin at an arbitrary depth d (d≥0) can be erased by adjusting the quantity of light having the first wavelength λ1 and the quantity of light having the second wavelength λ2, selected based on the wavelength selection method, so as to become equal on the surface (depth 0) of the object.

In some cases, the quantity of light having the second wavelength may be set to be higher than the quantity of light having the first wavelength in the adjustment of light quantity. If the purpose of adjustment is to erase the signals of hemoglobin, the signals of hemoglobin can be removed with greater certainty by setting the quantity of light having the second wavelength to be higher than the quantity of light having the first wavelength, without setting any limit to the quantity of light having the second wavelength.

The output of the light source may fluctuate somewhat from the set value due to the output characteristic of the light source. For example, if the fluctuation range of the output from the set value is ±q % (q is a positive value), it is very difficult to erase the signal of hemoglobin when the output of the light having the first wavelength λ1 increases q % and the output of the light having the second wavelength λ2 decreases q %. Therefore if the output of the light having the second wavelength is set to be higher than the output of the light having the first wavelength by (2×q) % or more in advance, then the signal of hemoglobin can be erased with certainty even if the output of the light source fluctuates. Typically it is preferable to control the light quantity so that Φ(λ1)=Φ(λ2)×(1+2q/100) is established.

For example, if the fluctuation range of the light quantity is ±5%, it is necessary for the quantity of light having the first wavelength λ1 to increase 5% and for the quantity of light having the second wavelength λ2 to decrease 5% to completely erase the signal of hemoglobin with certainty. In this case, it is preferable to adjust the set value of the quantity of light having the second wavelength to be higher than the set value of the quantity of light having the first wavelength by 10% or more (1.1 times or more).

(Adjusting Irradiation Light Quantity: Enhancing Contrast Agent)

Next the light quantity adjustment, in the case of detecting the contrast agent (e.g. ICG, ICG-PEG), will be described. If the first wavelength is 797 nm and the second wavelength is 850 nm, it is preferable to select the set value of the quantity of light having the second wavelength to 1.8 times or less the set value of the quantity of light having the first wavelength. Then even if the light quantity fluctuates 5%, the intensity of the signal generated by calculating the difference of signals imaged with the two wavelengths becomes a 90% or higher intensity of the signal of the contrast agent imaged with the first wavelength, because of the difference in the light absorption coefficient of the contrast agent.

If an OPO laser or a Ti: sa laser, which has an output characteristic in the near infrared region, is used as the light source, a wavelength at which output is the maximum is in the 750 nm to 800 nm range, and if the wavelength becomes longer than this, output decreases. In this case, a signal of hemoglobin remains as a result of the subtraction processing, since the relationship of Expression (7) is not established. However, if the light quantity is adjusted according to the above mentioned method, the signal of hemoglobin can be erased with certainty by the subtraction processing.

(Adjusting Irradiation Light Quantity: Concrete Method)

The light quantity can be adjusted using a method appropriate for the light source to be used. In the case of a laser, for example, the voltage value to be applied may be changed, and in the case of an LED, a signal to be input to the light source, such as a voltage value or current value, may be changed to control the light quantity.

In this case, it is preferable to install a shutter to shield light between the light source and the surface of the object, and adjust the quantity of light having the first wavelength and the quantity of light having the second wavelength in advance in a shutter-closed state before measurement, and store the control conditions, under which the same light quantity values are acquired, in a memory or the like in advance. Thereby the adjustment time can be reduced and the light having the first wavelength and the light having the second wavelength can be imaged consecutively.

For example, if the output of the light source fluctuates ±q %, it is preferable to set the control conditions such that the quantity of light having the second wavelength is higher than the quantity of light having the first wavelength by (2×q) %. In this case, the light having the first wavelength is output in the above mentioned shutter-closed state, and a part of the light is branched and measured by a light quantity meter or the like. Then the light source control values (e.g. voltage, current) to adjust the irradiation light quantity are stored in memory in association with the light quantity. Then adjustment is performed so that the quantity of light having the second wavelength becomes higher than the quantity of light having the first wavelength by (2×q) %, and the light source control values in this case are stored. For example, if the output of the light source fluctuates ±5% as a characteristic of the light source, the control values, with which the quantity of light having the second wavelength becomes 1.1 times the quantity of light having the first wavelength, are stored.

In actual imaging of an object, the shutter is opened and the control values of the light source, which were stored for each wavelength in advance, are used, whereby light having a plurality of wavelengths, for which light quantity has been appropriately adjusted respectively, can be radiated consecutively.

In other words, the object information acquiring apparatus according to this embodiment has: a light source configured to radiate the first light having a first wavelength λ1 and the second light having a second wavelength λ2; and a detection unit configured to detect an acoustic wave, which is generated from an object irradiated with the light from the light source, and convert the acoustic wave into a detection signal. The object information acquiring apparatus also has: a signal processing unit configured to acquire characteristic information of the inside of the object based on the detection signal; and a light intensity acquiring unit configured to acquire the intensity of the light radiated from the light source. The signal processing unit acquires the characteristic information by performing subtraction processing between a signal generated as a result of the first light being absorbed by hemoglobin and a signal generated as a result of the second light being absorbed by hemoglobin. Here the first wavelength is 780 nm or more and 810 nm or less, and the second wavelength is 840 nm or more and 920 nm or less. An adjustment is performed so that Φ(λ1)≤Φ(λ2) is satisfied, and the difference between Φ(λ1) and Φ(λ2) is within a predetermined range, where Φ(λ1) denotes the incident light intensity of the first light to the object, and Φ(λ2) denotes the incident light intensity of the second light to the object, which are acquired by the light intensity acquiring unit.

(Correction of Position Shift of Acquired Image)

The correction of a position shift of acquired images will be described next. The position of the object may be changed, due to body movement or the like, during imaging at the selected wavelengths λ1 and λ2. In this case, a position shift is generated between the images acquired at each wavelength. To accurately remove the signals of hemoglobin in the above mentioned subtraction processing, it is preferable that the positions of the images acquired at wavelengths λ1 and λ2 match as closely as possible during this processing. Therefore a position shift of the images acquired at each wavelength may be corrected before executing the subtraction processing. For the position shift correction method, a part of a characteristic image acquired at each wavelength λ1 and λ2 is selected and corrected so that the image acquired at each wavelength matches. An image acquired at each wavelength may be divided into a plurality of images, so that an image similar to each divided image acquired at λ1 is searched and extracted in the divided image acquired at λ2, and the position shift amount of the divided images is estimated to correct the positions.

In some cases the position shift may be more accurately corrected when an object, to which the contrast agent has been administered, is imaged. If the contrast agent is administered into an object and the object is imaged at the first wavelength λ1 and the second wavelength λ2, the signals of blood vessels (hemoglobin) and of the contrast agent are mainly generated in the image acquired at the wavelength λ1, and the signals of the blood vessels (hemoglobin) are mainly generated in the image acquired at the wavelength λ2. Here a case of setting a characteristic area where the blood vessels and the contrast agent are generated in the image acquired at the wavelength λ1, and searching and extracting an image similar to the image of this characteristic area from the image acquired at the wavelength λ2, is considered. In the image acquired at the first wavelength λ1, both the signal of the blood vessels and the signal of the contrast agent are generated in a mixed state, but it is difficult to discern the signal of the blood vessels and the signal of the contrast agent by this image acquired at the wavelength λ1 alone. This means that three states are possible in this characteristic area: only the signal of the blood vessels exists; only the signal of the contrast agent exists; and both of these signals coexist. If only the signal of the blood vessels exists, the image corresponding to this signal has a matching image in the image acquired at the second wavelength λ2, therefore the shift amount thereof can be calculated, whereby the position shift can be corrected. In the case where both the signal of the blood vessels and the signal of the contrast agent coexist as well, an image which is similar to the image of the blood vessels included in this image exists in the image acquired at the wavelength λ2, therefore this image is detected, and the position shift thereof can be corrected using the shift amount of these images. However, if only the signal of the contrast agent exists in the characteristic area which was set in the image acquired at the wavelength λ1, then the position shift cannot be corrected since a similar image cannot be detected in the image acquired at the wavelength λ2. To prevent this from occurring, a plurality of characteristic areas may be set in the image acquired at the wavelength λ1, so that signals similar to those in these characteristic areas are searched and extracted in the image acquired at the wavelength λ2. In this case, a characteristic area of which similarity is less than the predetermined similarity may be set so that this characteristic area is not used for the correction of the position shift.

A case of setting the characteristic area in the image acquired at the second wavelength λ2 will be described next. In the image acquired at the wavelength λ2, an image of mainly blood vessels originating in hemoglobin is generated. According to the present invention, the wavelength range, where the image of the blood vessels originating in hemoglobin can be removed by subtracting the image acquired at the wavelength λ2 from the image acquired at the wavelength λ1 (subtraction processing), is selected. In other words, an image of blood vessels acquired at the wavelength 12 always has a corresponding image of blood vessels in the image acquired at the wavelength λ1. Therefore if a characteristic area is set in an image acquired at the wavelength λ2, and an image similar to this image is searched in the image acquired at the wavelength λ1, then a similar image can always be extracted and the position shift can be corrected based on the shift amount of these similar images. To improve the accuracy of the position shift correction, a plurality of characteristic areas may be set in the image acquired at the wavelength λ2, so that images similar to these characteristic areas are searched and extracted in the image acquired at the wavelength λ1. Further, the entire image acquired at the wavelength λ2 may be divided into a plurality of images, so that an image similar to each divided image is searched and extracted in the image acquired at the wavelength λ1. In this case, a characteristic area of which similarity is less than the predetermined similarity may be set so that this characteristic area is not used for the correction of the position shift.

To correct the position shift between a first reconstructed image acquired at the first wavelength λ1 and a second reconstructed image acquired at the second wavelength λ2, it is preferable that an image similar to one or a plurality of characteristic areas set in the second reconstructed image, is searched and extracted in the first reconstructed image, then the shift amount is calculated and the position shift is corrected.

As described above, the characteristic area may be set in the image acquired at the wavelength λ1 or the characteristic area may be set in the image acquired at the wavelength λ2 for correcting the position shift, but it is preferable to set the characteristic area in the image acquired at the wavelength λ2, since the rate of detecting a similar image is higher, and as a result, the accuracy of the position shift correction becomes higher.

(Object Information Acquiring Method)

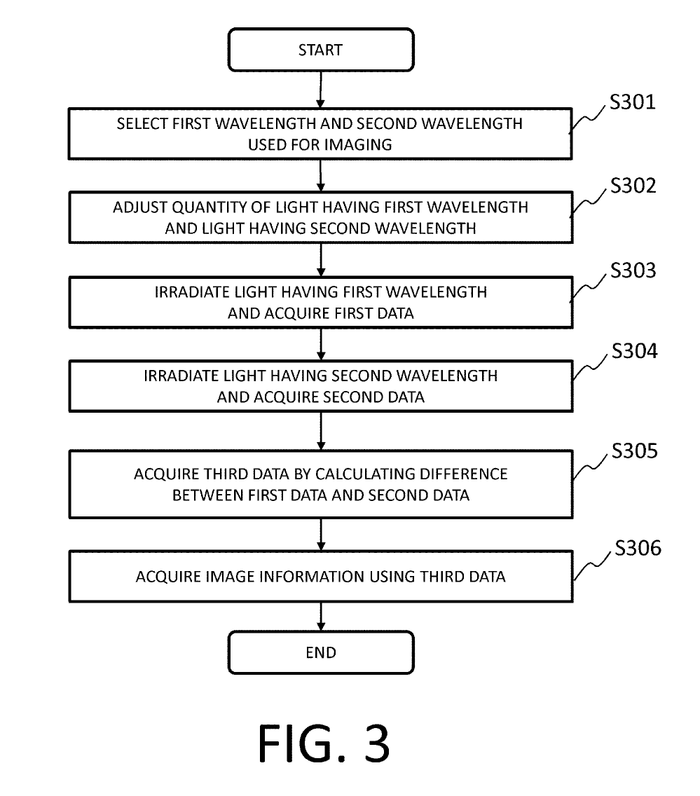

The processing performed by the signal processor 19 will be described with reference to FIGS. 3, 4 and 5. The step number in the description on each processing corresponds to the flow chart in FIG. 3.

Processing 1 (S301): step of selecting first wavelength and second wavelength used for imaging

Initially the first wavelength λ1 and the second wavelength λ2 of the light to be radiated into the object 15 are selected according to the above mentioned wavelength selection method.

Processing 2 (S302): step of adjusting light quantity of the first light and second light

Then before actually measuring the object 15, the light quantity of the first light and the second light are adjusted according to the above mentioned adjusting method. Thereby appropriate light source control conditions at each wavelength are acquired.

Processing 3 (S303): step of radiating first light and acquiring first data

Then the first light, of which light quantity has been adjusted, is radiated from the light source 11 into the object 15. The acoustic wave detector 17 acquires a first detection signal P1(t), and saves this signal in the memory of the signal processor 19 as first data.

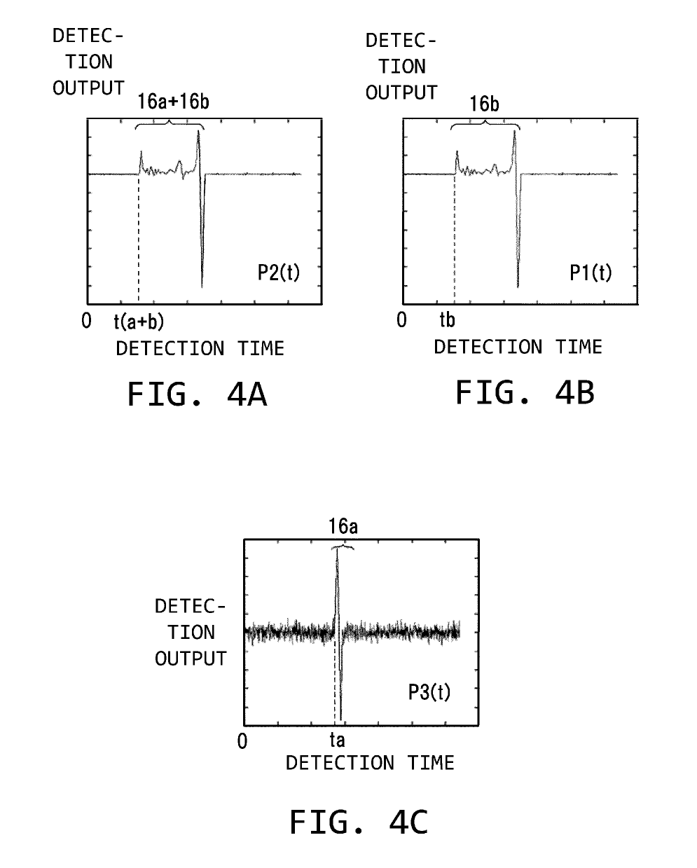

The first detection signal P1(t) acquired here will be described. FIG. 4A shows an example of the first detection signal P1(t), which was detected by a specific detection element and stored in the memory of a PC according to this step. In FIG. 4A, the abscissa indicates the detection time, where the time when the light irradiation is executed is 0. The ordinate indicates a value in proportion to the sound pressure detected by the acoustic wave detector 17.

Now a case when a contrast agent, which was a light absorption characteristic at the selected first wavelength λ1, exists inside the object, is considered. In this case, the acoustic detector detects both an acoustic wave (16b) generated from such a light absorber as blood vessels and hemoglobin, and an acoustic wave (16a) generated from a light absorber 101 originating in the contrast agent. Here the time t (a+b) is determined by dividing the shortest distance between the acoustic wave detector 17 and the signal transmitting portion (of one of the light absorbers) inside the object, when the acoustic wave detector 17 first detected a signal at the radiation of the first light, by an average sound velocity of the acoustic wave inside the object.

A case when the shortest distance db, between the acoustic wave detector 17 and the light absorber 14 originating in the hemoglobin, becomes approximately the same as the shortest distance da between the acoustic wave detector 17 and the light absorber 101 originating in the contrast agent, as shown in FIG. 1, is now considered. In this case, as shown in FIG. 4A, the acoustic wave 16a from the light absorber 101 originating in the contrast agent and the acoustic wave 16b from the light absorber 14 originating in hemoglobin are detected almost at the same time. Therefore the detection signal, detected by the acoustic wave detector 17, is a superimposed signal of the acoustic wave 16a and the acoustic wave 16b.

FIG. 4B shows a signal when the acoustic wave 16b, generated from the light absorber 14 originating in the hemoglobin inside the object, is detected. No major difference is observed between the signals in FIG. 4A and FIG. 4B. That is, from the detection signal shown in FIG. 4A in this case, it is difficult to distinguish between the detection signal of the acoustic wave 16b generated from the light absorber 14 originating in hemoglobin and the detection signal of the acoustic wave 16a generated from the light absorber 101 originating in the contrast agent.

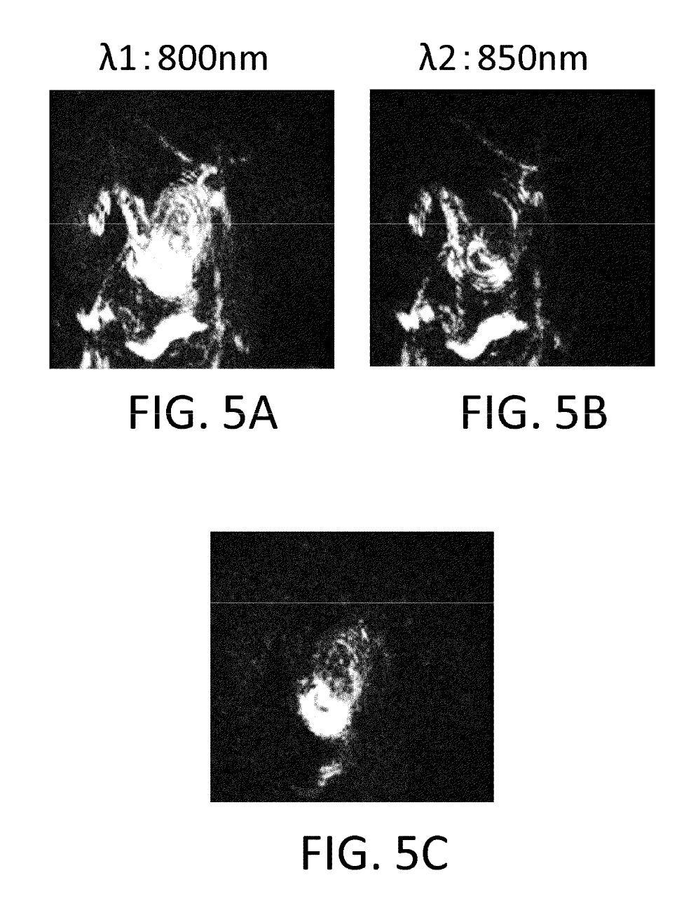

In the above description, the first data is the first detection signal P1(t), but the first data may be first image information T1(r) which is acquired by performing image reconstruction processing using the first detection signal P1(t). In this case, the first image information T1(r), related to the optical characteristic value distribution of the object, is generated by performing the image reconstruction processing using the first detection signal P1(t), and is saved in the memory of a PC, which is the signal processor 19. FIG. 5A is an example of the first image information T1(r) acquired by performing the image reconstruction using the first detection signal P1(t). FIG. 5A is an image captured after a contrast agent is administered into a cancerous mouse. In a high contrast region (white region) in FIG. 5A, images of blood vessels originating in the hemoglobin in the body of the mouse and images originating in the contrast agent drawn to a cancer coexist.

Processing 4 (S304): step of radiating second light and acquiring second data

Then the second light is radiated into the object 15 using the adjusted light quantity. The acoustic wave detector 17 acquires the second detection signal P2(t), and saves it as the second data in the memory of the signal processor 19.

It is possible that a contrast agent, of which light absorption coefficient at the wavelength λ2 is smaller than the light absorption coefficient at the wavelength λ1, is used. In this case, an acquired signal is a mixture of the signal originating in the hemoglobin of which level is about the same as or higher than the signal originating in the hemoglobin when the first light is radiated, and the signal originating in the contrast agent of which level is lower than the signal originating in the contrast agent when the first light having the wavelength λ1 is radiated (FIG. 4B). Here the time tb is the time when the acoustic wave detector 17 detected the signal for the first time when the second light is radiated, and is determined by dividing the shortest distance between the acoustic wave detector 17 and the signal transmitting part in the object by the average sound velocity of the acoustic wave in the object. If a wavelength, with which light absorption of the contrast agent does not occur, is selected as the wavelength λ2 of the second light, then only the signal originating in the hemoglobin can be acquired.

In the above description, the second data is the second detection signal P2(t), but the second data may be the second image information T2(r) which is acquired by performing image reconstruction using the second detection signal P2(t). In this case, the image reconstruction processing is performed using the second detection signal P2(t), the second image information T2(r) related to the optical characteristic value distribution of the object is generated, and this second image information T2(r) is saved in the memory of the signal processor 19. FIG. 5B is an example of the second image information T(r) that is acquired by performing the image reconstruction using the second detection signal P2(t). In FIG. 5B, unlike FIG. 5A, mainly the light absorber 14 originating in hemoglobin is imaged.

One method of acquiring a detection signal by scanning the acoustic wave detector is acquiring a detection signal by scanning the acoustic wave detector with fixing the wavelength of the irradiation light to λ1, and then acquiring a detection signal by scanning the acoustic wave detector in the same manner with fixing the wavelength of the light to λ2. This method is preferable since a number of times of switching the wavelengths of the light source can be minimal, that is, the load on the light source is low.

Another scanning method is repeating the process of acquiring both the detection signals P1 and P2 by radiating the light having the wavelength λ1 and light having the wavelength λ2 at a certain measurement position, and acquiring the detection signals P1 and P2 in the same manner at the next measurement position. This method is preferable since a position shift does not occur very much when the detection signals P1 and P2 are acquired.

Processing 5 (S305): step of acquiring third data by calculating difference between first data and second data

Then a third detection signal P3(t) is acquired as the third data, using the first detection signal P1(t) and the second detection signal P2(t) which were saved in the signal processor 19 in S303 and S304. Here a difference signal is calculated by subtracting P2(t) from P1(t), whereby P3(t) is acquired. As a result, a signal, as shown in FIG. 4C, for example, is acquired.

FIG. 4 shows the result of subtracting the second detection signal P2(t) from the first detection signal P1(t), where a signal, caused by the acoustic wave 16a generated from the light absorber 101 originating in the contrast agent inside the object, is reproduced within the detection signal. Therefore the detection signal, caused by the acoustic wave 16b generated from the light absorber 14, originating in the hemoglobin, and the detection signal, caused by the acoustic wave 16a generated from the light absorber 101 originating in the contrast agent, can be distinguished, although this was impossible in FIG. 4A.

As described above, in this embodiment, the new third detection signal P3(t) is acquired from the first detection signal P1(t) and the second detection signal P2(t) corresponding to each wavelength. Thereby the detection signal, caused by the acoustic wave 16b originating in the hemoglobin, can be erased, and the detection signal, caused by the acoustic wave 16a generated from the light absorber 101 originating in the contrast agent inside the object, can be extracted. Here the time to is determined by dividing the distance da between the acoustic wave detector 17 and the light absorber 101, originating in the contrast agent inside the object, by the average sound velocity of the acoustic wave inside the object.

In the above description, the third data is the third detection signal P3(t). However the third data may be the third image information T3(r) acquired from the first image information T1(r) and the second image information T2(r). In this case, the third image information T3(r), as the third data, is acquired by calculating the different image information, that is, by subtracting the second image information T2(r) from the first image information T1(r). The step sequence of Processing 3 and Processing 4 may be reversed.

If a position shift is generated between the first image information T1(r) and the second image information T2(r), a step to correct this shift is added. In this case, a characteristic area where the blood vessels are imaged is set in the second image information T2(r), and an image similar to this characteristic area is searched and extracted in the first image information T1(r). The second image information T2(r) may be divided in the same manner, so that an image similar to the first image information T1(r) divided in the same manner may be search and extracted in the image information T2(r), and the position shift is corrected based on this shift amount.

Processing 6 (S306): step of generating image information using third data

When the first data and second data are the first detection signal P1(t) and the second detection signal P2(t) respectively, the image reconstruction processing is performed using the third detection signal P3(t) which was acquired as the third data in S303, whereby the third image information T(r) is generated. As shown in FIG. 4C, the third data is a signal where the detection signal of the acoustic wave generated in the light absorber 14 originating in the hemoglobin has been erased. Therefore mainly the light absorber 101, originating in the contrast agent inside the object, can be imaged. FIG. 5C shows an example of image information that is acquired as a result of this processing. The high contrast region (white region) in FIG. 5C is an image of the light absorber 101 originating in the contrast agent inside the object. If the first data and second data are the first image information and second image information respectively, processing 6 is unnecessary.

By performing the above mentioned steps, the signals originating in hemoglobin can be erased, even in photoacoustic imaging where an oxidation or deoxidation state of hemoglobin at an arbitrary depth from the surface of the object cannot be predicted in advance. As a result, an image, where only the signal originating in the contrast agent is extracted, can be acquired for a minimal number of measurement times. The program, including the above mentioned steps, may be executed by the signal processor 19, which functions as a computer.

One method (method A) for acquiring the reconstructed image is acquiring the third detection signal P3(t) from the difference between the detection signal P1(t) and the detection signal P2(t). Another method (method B) is acquiring the third image information T3(r) from the difference between the first image information T1(r) and the second image information T2(r). The characteristics of each method will be described.

In the case of method A, the image reconstruction is performed only once, hence it does not take much time for the image reconstruction, and the calculation processing load is low. It is preferable that the detection signal P1(t) acquired by radiating the light having the wavelength λ1 and the detection signal P2(t) acquired by radiating the light having the wavelength λ2 are acquired at a same position. If a position shifts between the radiation of the light having the wavelength λ1 and the radiation of the light having the wavelength λ2, it is preferable to correct the position shift considering this shift. For example, the coordinates of each measured position, at which the light having the wavelength λ1 or the wavelength λ2 is radiated, is stored in advance, and the coordinates of each position are compared. If there is a difference, then position shift correction is performed according to this shift amount.

In the case of method B, three types of information are acquired: first image information T1(r) and second image information T2(r), based on the region where the blood vessels and the contrast agent exist, can be displayed, and third image information T3(r), based on the region where the contrast agent exists, can be displayed. Therefore merely by displaying these three types of image information to the user, the user can recognize not only the position of the contrast agent (position where a tumor is likely to exist), but also the position of the blood vessels, and the relative positional relationship between the contrast agent and the blood vessels. To display the image information to the user, the first to third image information may be displayed all at once, or may be displayed in the sequence of the acquisition of the information, or in any other order.

In the case of method B, the image reconstruction is performed twice, hence it takes time to acquire the image. However the time to acquire the third image information can be shortened if the image reconstruction processing to acquire the first image information T1(r) is executed while receiving the detection signal to acquire the second image information T2(r). To perform this parallel processing, it is preferable that the object information acquiring apparatus has both a processor to acquire the detection signal and a processor to reconstruct the image.

In other words, the object information acquiring method according to this embodiment has the following steps:

(1) a step of radiating the first light having the first wavelength λ1 and the second light having the second wavelength λ2 from the light source,
(2) a step of the detection unit detecting an acoustic wave generated from the object irradiated with the light from the light source, and converting the acoustic wave into a detection signal,
(3) a step of the signal processing unit acquiring the characteristic information of the inside of the object based on the detection signal,
(4) a step of acquiring the intensity of light radiated from the light source, and
(5) a step of performing an adjustment so that Φ(λ1) Φ(λ2) is satisfied, and the difference between Φ(λ1) and Φ(λ2) is within a predetermined range, where Φ(λ1) denotes the incident light intensity of the first light to the object, and Φ(λ2) denotes the incident light intensity of the second light to the object.

Here the first wavelength is 780 nm or more and 810 nm or less, and the second wavelength is 840 nm or more and 920 nm or less.

In the step of acquiring the intensity of the light, the signal processing unit acquires the characteristic information by performing subtraction processing between a signal generated as a result of the first light being absorbed by hemoglobin and a signal generated as a result of the second light being absorbed by the hemoglobin.

If the characteristic area is set in the second image information T2(r) and an image similar to this [characteristic area] is searched and extracted in the first image information T1(r) for correcting the position shift, it is preferable that imaging to acquire the first detection signal P1(t) is performed while acquiring the second image information T2(r), and the first detection signal P1(t) is reconstructed and the image information T1(r) is acquired while setting the characteristic area in the second image information T2(r).

In the case of subtracting the second reconstructed image from the first reconstructed image, after the position shift is corrected between the first reconstructed image and the second reconstructed image, it is preferable to include: a step of setting a characteristic area in the second reconstructed image; a step of searching and extracting an image similar to this characteristic area in the first reconstructed image; and a step of calculating the shift amount from the extracted image and performing position correction.

### Configuration Example

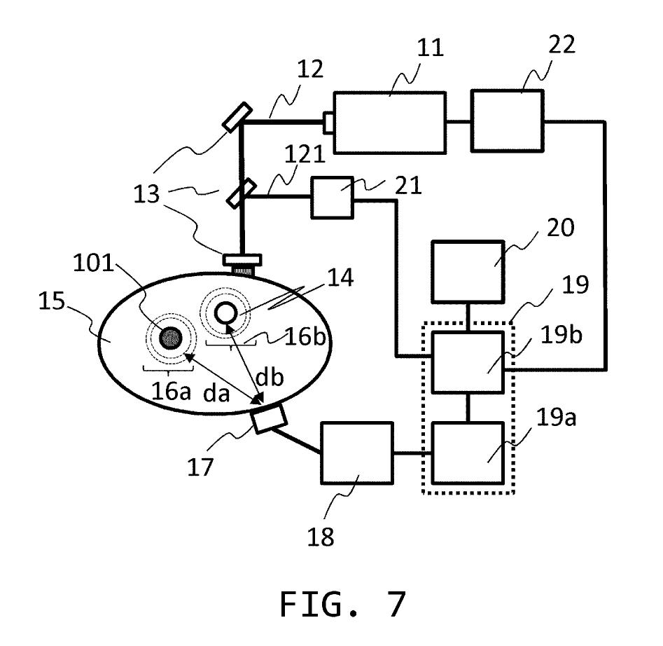

An example of the apparatus configuration, which is preferable to carry out the present invention, will now be described with a reference to FIG. 7.

(Light Source 11 and Light Source Unit 22)

The light source 11 can radiate light having at least two different wavelengths. In the wavelength region that can be output, it is assumed that the light absorption coefficients of the oxyhemoglobin and deoxyhemoglobin satisfy the following condition,

μ_{HbO2}(λ1)≤μ_{HbO2}(λ2) and μ_{Hb}(λ1)≤μ_{Hb}(λ2)

where λ1 and λ2 denote the two wavelengths. In concrete terms, it is preferable that light having two different wavelengths in the 780 nm to 920 wavelength range can be output. Further, it is preferable that the wavelength can be selected and output consecutively in this wavelength range.

The light source unit 22 adjusts the wavelength and the light quantity of the light radiated from the light source 11. To control the light quantity, the electric signal (current, voltage) to be applied to the light source is controlled. In some cases, the light source may include functions to control the wavelength and the light quantity. The light source unit 22 may control the irradiation timing, the waveform and the intensity of the irradiation light. The light source and the light source unit of this embodiment may be integrated with the object information acquiring apparatus of this embodiment, or the light source may be a standalone device.

For the light source 11, a pulsed light source, that can generate a pulsed light at a several nano to several hundred nanosecond order, is preferable. In concrete terms, about a 10 nanosecond pulse width is used to efficiently generate an acoustic wave. For the light source, laser is preferable because of the high output. However, a light emitting diode, flash lamp or the like may be used. For laser, various types of lasers can be used, such as a solid-state laser, a gas laser, a dye laser and a semiconductor laser. The laser may be constituted by a plurality of lasers. For example, an OPO laser or a dye laser excited by a YAG laser, or a Ti: sa laser can be used.

(Optical System 13)

The optical system 13 guides the light 12 radiated from the light source 11 to the object while processing the light into a desired light distribution shape. The optical system 13 includes, for example, a mirror to reflect light, lenses to collect or expand light or to change the shape of the light, a diffusion plate to diffuse light, and an optical fiber. Any of these optical components can be used as long as the light 12, emitted from the light source, can be radiated into the object 15 with a desired shape. It is preferable that the light is expanded to a certain area, instead of being collected by a lens, due to safety concerns for living bodies and the expansion of the diagnostic region. A shutter to shield the light may be installed between the light source and the surface of the object.

(Light Absorber 14 and Object 15)

These are not a part of the apparatus of this embodiment, nonetheless both will be described herein below. The object information acquiring apparatus of this embodiment is used mainly for the purpose of the diagnosis of malignant tumors and vascular diseases of humans and animals, and for the follow up observation of chemotherapy. Therefore the assumed object 15 is a living body, more specifically, such diagnostic target objects as the breasts, fingers and limbs of humans and animals. The light absorber 14 existing inside the object is, for example, oxyhemoglobin, deoxyhemoglobin and blood vessels that contain a large amount of oxyhemoglobin and hemoglobin.

(Light Absorber 101: Contrast Agent)

Now a case of administering such a light absorber as a contrast agent into an object will be described. In this embodiment, it is desired to erase the signal from the light absorber originating in hemoglobin, and acquire only the signal of the contrast agent. Therefore PAI is performed with selecting two wavelengths by the above mentioned wavelength selection method, so as to select a contrast agent having a light absorption coefficient, with which the signal from the contrast agent is not erased when the signals acquired at respective wavelengths are subtracted.

In other words, if the light absorption coefficient of the selected contrast agent is μ_{CA}(λ), the light absorption coefficient μ_{CA}(λ1) at the first wavelength λ1 and the light absorption coefficient μ_{CA}(λ2) at the second wavelength λ2 have the following relationship in the above mentioned wavelength range.

μ_{CA}(λ1)>μ_{CA}(λ2)

Because of this relationship, a positive signal is always acquired, even if the signal at the second wavelength λ2 is subtracted from the signal at the first wavelength λ1. As a result, only a signal originating in the contrast agent, where a signal originating in hemoglobin has been erased, can be acquired. It is preferable to select two wavelengths, with which the difference between μ_{CA}(λ1) and μ_{CA}(λ2) becomes the maximum. Thereby the signal of the contrast agent after subtracting the signals at each wavelength can be maximized, hence a clear signal originating in the contrast agent can be acquired.

In this description, the contrast agent refers to a light absorber that is externally administered into the object mainly for improving the contrast (SN ratio) of the photoacoustic signal distribution. Besides the light absorber itself, the contrast agent can contain a material for controlling internal kinetics. The material for controlling internal kinetics is, for example, a serum-derived protein (e.g. albumin, IgG) and a water-soluble synthetic polymer (e.g. polyethylene glycol). Therefore the contrast agent in this description includes, a solo light absorber, a covalent bond of a light absorber and other materials, and a light absorber and other materials which are held together by physical interaction. If the contrast agent has a function to be drawn specifically to a malignant tumor of a human or animal, then a signal from the tumor can be acquired in PAI from the contrast agent.

If the object is a living body, near-infrared (wavelength: 600 nm to 900 nm) is preferable as the irradiation light, in terms of safety of and transmissivity through a living body. Therefore a material having a light absorption characteristic at least in the near-infrared wavelength region is used for the contrast agent. For example, a cyanine compound (also called “cyanine pigment”) represented by indocyanine green, and an inorganic compound represented by gold and iron oxide, can be used. The cyanine compound in this embodiment preferably has a molar absorption coefficient 10⁶ M⁻¹ cm⁻¹ or more at the maximum absorption wavelength. Examples of the structure of the cyanine compound of this embodiment are expressed by the following General Formulas (1) to (4).

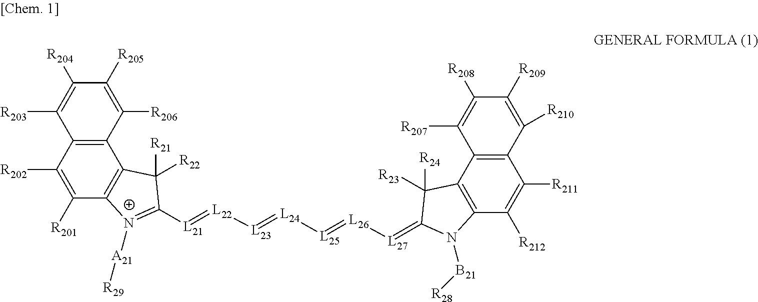

In General Formula (1), R₂₀₁ to R₂₁₂ are a mutually-independent hydrogen atom, halogen atom, SO₃T₂₀₁, PO₃T₂₀₁, benzene ring, thiophene ring, pyridine ring, or a straight chain or branched alkyl group of which carbon number is 1 to 18. The above mentioned T₂₀₁ is one of: a hydrogen atom, sodium atom and potassium atom. In General Formula (1), R₂₁ to R₂₄ are a mutually-independent hydrogen atom, or a straight chain or branched alkyl group of which carbon number is 1 to 18. In General Formula (1), A₂₁ and B₂₁ are a mutually-independent straight chain or branched alkylene group of which carbon number is 1 to 18. In General Formula (1), L₂₁ to L₂₇ are a mutually-independent CH or CR₂₅. The above mentioned R₂₅ is a straight chain or branched alkyl group of which carbon number is 1 to 18, halogen atom, benzene ring, pyridine ring, benzyl group, ST₂₀₂ or a straight chain or branched alkylene group of which carbon number is 1 to 18. The above mentioned T₂₀₂ is a straight chain or branched alkyl group of which carbon number is 1 to 18, benzene ring, or a straight chain or branched alkylene group of which carbon number is 1 to 18. In General Formula (1), L₂₁ to L₂₇ may form from a four-member ring to a six-member ring. In General Formula (1), R₂₈ is one of: —H, —OCH₃, —NH₂, —OH, —CO₂T₂₈, —S(═O)₂OT₂₈, —P(═O)(OT₂₈)₂, —CONH—CH(CO₂T₂₈)—CH₂(C═O)OT₂₈, —CONH—CH(CO₂T₂₈)—CH₂CH₂ (C═O)OT₂₈, and —OP(═O)(OT₂₈)₂. The above T₂₈ is one of: a hydrogen atom, sodium atom and potassium atom. In General Formula (1), R₂₉ is one of: —H, —OCH₃, —NH₂, —OH, —CO₂T₂₉, —S(═O)₂OT₂₉, —P(═O)(OT₂₉)₂, —CONH—CH(CO₂T₂₉)—CH₂(C═O)OT₂₉, —CONH—CH(CO₂T₂₉)—CH₂CH₂(C═O)OT₂₉, and —OP(═O)(OT₂₉)₂. The above T₂₉ is one of: a hydrogen atom, sodium atom and potassium atom.

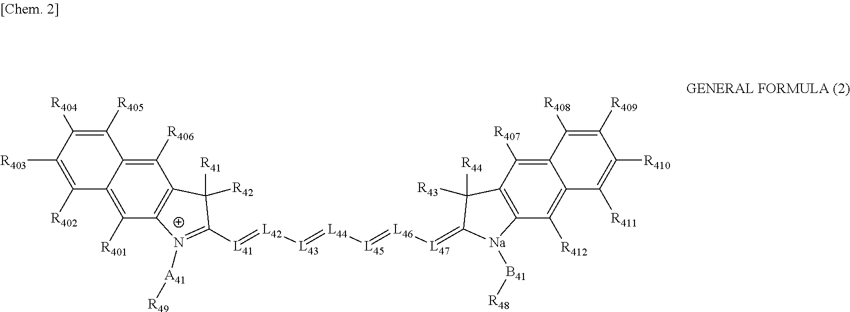

In General Formula (2), R₄₀₁ to R₄₁₂ are a mutually-independent hydrogen atom, halogen atom, SO₃T₄₀₁, PO₃T₄₀₁, benzene ring, thiophene ring, pyridine ring, or a straight chain or branched alkyl group of which carbon number is 1 to 18. The above mentioned T₄₀₁ is one of: a hydrogen atom, sodium atom and potassium atom. In General Formula (2), R₄₁ to R₄₄ are a mutually-independent hydrogen atom, or a straight chain or branched alkyl group of which carbon number is 1 to 18. In General Formula (2), A₄₁ and B₄₁ are a mutually-independent straight chain or branched alkylene group of which carbon number is 1 to 18. In General Formula (2), L₄₁ to L₄₇ are a mutually-independent CH or CR₄₅. The above mentioned R₄₅ is a straight chain or branched alkyl group of which carbon number is 1 to 18, halogen atom, benzene ring, pyridine ring, benzyl group, ST₄₀₂ or a straight chain or branched alkylene group of which carbon number is 1 to 18. The above mentioned T₄₀₂ is a straight chain or branched alkyl group of which carbon number is 1 to 18, benzene ring, or straight chain or branched alkylene group of which carbon number is 1 to 18. In General Formula (2), L₄₁ to L₄₇ may form a four-member ring or six-member ring. In General Formula (2), R₄₈ is one of: —H, —OCH₃, —NH₂, —OH, —CO₂T₄₈, —S(═O)₂OT₄₈, —P(═O)(OT₄₈)₂, —CONH—CH(CO₂T₄₈)—CH₂(C═O)OT₄₈, —CONH—CH(CO₂T₄₈)—CH₂CH₂ (C═O)OT₄₈, and —OP(═O)₂(OT₄₈)₂. The above mentioned T₄₈ is one of: a hydrogen atom, sodium atom and potassium atom. In General Formula (2), R₄₉ is one of: —H, —OCH₃, —NH₂, —OH, —CO₂T₄₉, —S(═O)₂OT₄₉, —P(═O)(OT₄₉)₂, —CONH—CH(CO₂T₄₉)—CH₂(C═O)OT₄₉, —CONH—CH(CO₂T₄₉)—CH₂CH₂(C═O)OT₄₉, and —OP(═O)(OT₄₉)₂. The above mentioned T₄₉ is one of: a hydrogen atom, sodium atom and potassium atom.

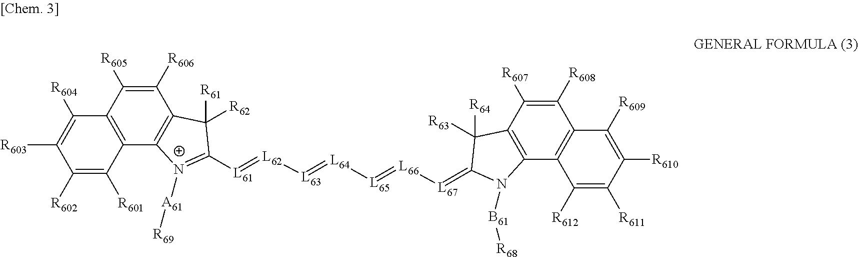

In General Formula (3), R₆₀₁ to R₆₁₂ are a mutually-independent hydrogen atom, halogen atom, SO₃T₆₀₁, PO₃T₆₀₁, benzene ring, thiophene ring, pyridine ring, or a straight chain or branch alkyl group of which carbon number is 1 to 18. The above mentioned T₆₀₁ is one of: a hydrogen atom, sodium atom and potassium atom. In General Formula (3), R₆₁ to R₆₄ are a mutually-independent hydrogen atom, or a straight chain or branched alkyl group of which carbon number is 1 to 18. In General Formula (3), A₆₁ and B₆₁ are a mutually-independent straight chain or branched alkylene group of which carbon number is 1 to 18. In General Formula (3), L₆₁ to L₆₇ are a mutually-independent CH or CR₆₅. The above mentioned R₆₅ is a straight chain or branched alkyl group of which carbon number is 1 to 18, halogen atom, benzene ring, pyridine ring, benzyl group, ST₆₀₂ or a straight chain or branched alkylene group of which carbon number is 1 to 18. The above mentioned T₆₀₂ is a straight chain or branched alkyl group of which carbon number is 1 to 18, benzene ring, or a straight chain or branched alkylene group of which carbon number is 1 to 18. In General Formula (3), L₆₁ to L₆₇ may form a four-member ring or six-member ring. In General Formula (3), R₆₈ is one of: —H, —OCH₃, —NH₂, —OH, —CO₂T₆₈, —S(═O)₂OT_{H}, —P(═O)(OT₆₈)₂, —CONH—CH(CO₂T₆₈)—CH₂(C═O)OT₆₈, —CONH—CH(CO₂T₆₈)—CH₂CH₂(C═O)OT₆₈, and —OP(═O)(OT₆₈)₂. The above mentioned T₆₈ is one of: a hydrogen atom, sodium atom and potassium atom. In General Formula (3), R₆₉ is one of: —H, —OCH₃, —NH₂, —OH, —CO₂T₆₉, —S(═O)₂OT₆₉, —P(═O)(OT₆₉)₂, —CONH—CH(CO₂T₆₉)—CH₂ (C═O)OT₆₉, —CONH—CH(CO₂T₆₉)—CH₂CH₂ (C═O)OT₆₉, and —OP(═O)(OT₆₉)₂. The above mentioned T₆₉ is one of: a hydrogen atom, sodium atom and potassium atom.

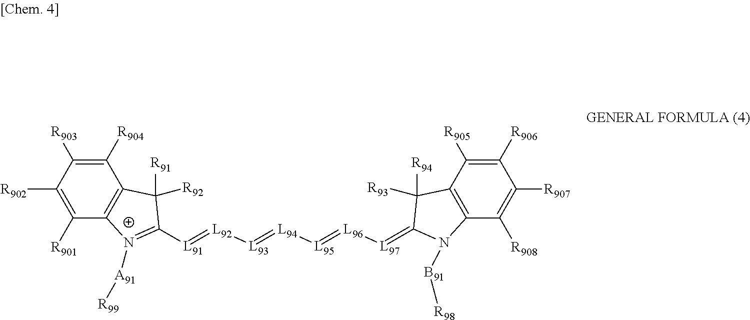

In General Formula (4), R₉₀₁ to R₉₀₈ are a mutually-independent hydrogen atom, halogen atom, SO₃T₉₀₁, PO₃T₉₀₁, benzene ring, thiophene ring, pyridine ring, or a straight chain or branched alkyl group of which carbon number is 1 to 18. The above mentioned T₉₀₁ is one of: a hydrogen atom, sodium atom and potassium atom. In General Formula (4), R₉₁ to R₉₄ are a mutually-independent hydrogen atom, or a straight chain or branched alkyl group of which carbon number is 1 to 18. In General Formula (4), A₉₁ and B₉₁ are a mutually-independent straight chain or branched alkylene group of which carbon number is 1 to 18. In General Formula (4), L₉₁ to L₉₇ are a mutually-dependent CH or CR₉₅. The above mentioned R₉₅ is a straight chain or branched alkyl group of which carbon number is 1 to 18, halogen atom, benzene ring, pyridine ring, benzyl group, ST₉₀₂ or straight chain or branched alkylene group of which carbon number is 1 to 18. The above mentioned T₉₀₂ is a straight chain or branched alkyl group of which carbon number is 1 to 18, benzene ring, or straight chain or branched alkylene group of which carbon number is 1 to 18. In General Formula (4), L₉₁ to L₉₇ may form a four-member ring or six-member ring. In General Formula (4), R₉₈ is one of: —H, —OCH₃, —NH₂, —OH, —CO₂T₉₈, —S(═O)₂OT₉₈, —P(═O)(OT₉₈)₂, —CONH—CH(CO₂T₉₈)—CH₂(C═O)OT₉₈, —CONH—CH(CO₂T₉₈)—CH₂CH₂ (C═O)OT₉₈, and —OP(═O) (OT₉₈)₂. The above mentioned T₉₈ is one of: a hydrogen atom, sodium atom and potassium atom. In General Formula (4), R₉₉ is one of: —H, —OCH₃, —NH₂, —OH, —CO₂T₉₉, —S(═O)₂OT₉₉, —P(═O)(OT₉₉)₂, —CONH—CH(CO₂T₉₉)—CH₂ (C═O)OT₉₉, —CONH—CH(CO₂T₉₉)—CH₂CH₂(C═O)OT₉₉, and —OP(═O)(OT₉₉)₂. The above mentioned T₉₉ is one of: a hydrogen atom, sodium atom and potassium atom.

Examples of the cyanine compound of this embodiment are indocyanine green, SF-64 having a benzo tricarbocyanine structure expressed by Chemical Formula 1, and the compounds expressed by Chemical Formula (i) to Chemical Formula (v) follow.

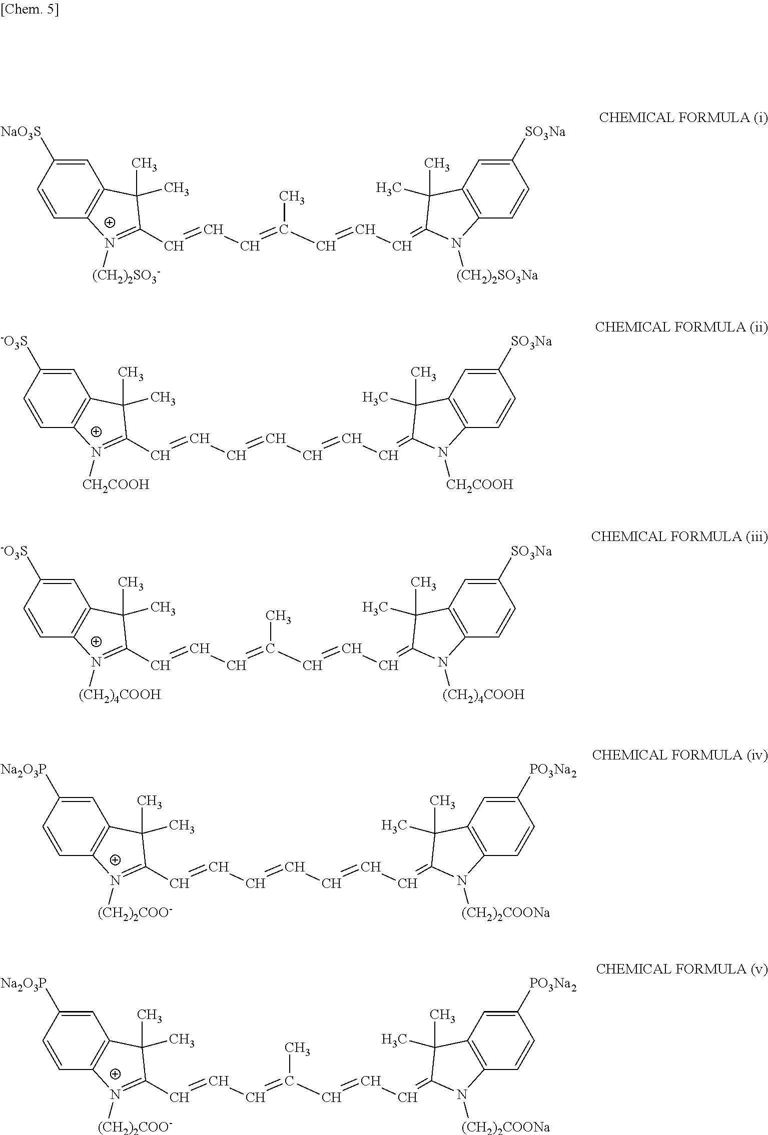

In the above mentioned cyanine compound, the aromatic ring may be substituted with a sulfonate group, carboxyl group or phosphate group. Further, a sulfonate group, a carboxyl group or a phosphate group may be introduced to a portion other than the aromatic ring. Examples of the contrast agent are: conjugate of indocyanine green and polyethylene glycol (ICG-PEG), conjugate of indocyanine green and human serum albumin (ICG-HSA), and liposome including indocyanine green. Here indocyanine green, polyethylene glycol and human serum albumin include the respective derivatives.

The contrast agent of this embodiment may contain a physiological saline, distilled water for injection, phosphate-buffered physiological saline, ringer solution, glucose solution or the like as the dispersion medium. The substance contained in the contrast agent may be dispersed in the dispersion medium in advance, or the substance and the dispersion medium may be prepared as a kit, so that the substance can be dispersed in the dispersion medium before administering the contrast agent into the living body. The contrast agent of this embodiment may further contain pharmacologically acceptable additives, such as a diluting agent, vasodilator, pH regulator, isotonizing agent, stabilizer and solubilizing agent. The contrast agent for optical imaging according to this embodiment may include an additive used for freezing and drying. Examples of this additive include: glucose, lactose, mannitol, polyethylene glycol, glycine, sodium chloride and sodium hydrogen phosphate. Only one type of additive may be used, or a plurality of types of additives may be used.

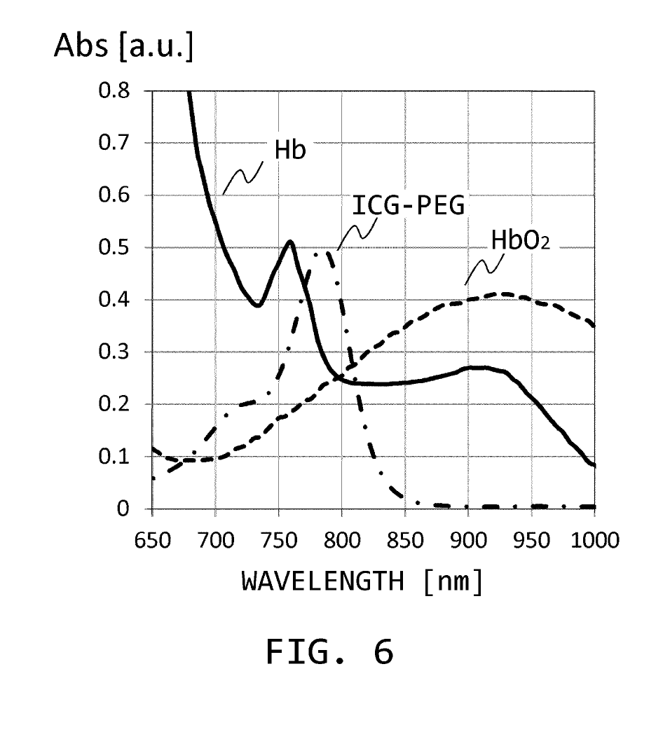

As an example, FIG. 6 shows the light absorption characteristics of ICG-PEG (dashed line). FIG. 6 also shows the light absorption characteristics of HbO₂ (broken line) and Hb (solid line).

A contrast agent is acceptable if the following relationship is satisfied.

μ_{CA}(λ1)>μ_{CA}(λ2)

When the light absorption characteristics satisfy the above mentioned conditions:

μ_{HbO2}(λ1)≤μ_{HbO2}(λ2), μ_{Hb}(λ1)≤μ_{Hb}(λ2), λ1<λ2

In other words, the contrast agent is not limited to a contrast agent containing indocyanine green or an indocyanine green derivative. For the administering means and method of the contrast agent, any known apparatus and method can be used, typically via blood vessels.

(Acoustic Wave Detector 17)

The acoustic wave detector 17 detects an acoustic wave that is generated on the surface of the object and inside the object by pulsed light, and converts the acoustic wave into an electric signal, which is an analog signal. The acoustic wave detector is also called a “probe” or “transducer”. Any transducer may be used as long as the acoustic wave signal can be detected, such as a transducer using the piezoelectric phenomenon, a transducer using the resonance of light, and a transducer using the change of capacitance.

The acoustic wave detector 17 of this embodiment preferably has a plurality of detection elements that are arrayed one-dimensionally or two-dimensionally. If multi-dimensionally arrayed elements are used in this way, an acoustic wave can be detected simultaneously at a plurality of locations, where decreasing the detection time, reducing the influence of object vibration, improving the SN ratio and the like can be expected.

An acoustic wave detector having a plurality of detection elements, which are disposed on the inner surface of a bowl-shaped or spherical crown-shaped support member, may be used. In this case, the plurality of detection elements are disposed such that a region, where the high reception sensitivity directions (directional axes) of at least a part of the detection elements concentrate, is generated. Thereby a high sensitivity region, in which the inside of the object can be imaged at high definition, can be generated. A light emission end may be set near the center of such a support member.

(Scanning Unit)

A scanning unit configured to change the relative position of the acoustic wave detector, with respect to the object, may be disposed. Thereby image data for a wide range of the object can be generated. The scanning unit may move the light emission end of the optical system synchronizing with the acoustic wave detector. If the object is held by a plate member, the acoustic wave detector can be moved along the plate surface. If the object is held by a cup-shaped member, the acoustic wave detector can be moved within a plane below the object.

(Signal Collector 18)

The signal collector 18 performs such processing as amplification, A/D conversion and correction for an electric signal output from the acoustic wave detector 17. The signal collector 18 is typically constituted by an amplifier, an A/D convertor, an FPGA (Field Programmable Gate Array) chip and the like. If a plurality of detection signals are acquired from the acoustic wave detector 17, it is preferable to process the plurality of signals simultaneously. Thereby the time to generate an image can be decreased.

(Signal Processor 19)

The signal processor 19 performs reduction processing to reduce the acoustic wave signal generated on the surface of the object, which is characteristic processing of this embodiment. Then the signal processor 19 reconstructs the image using a new signal generated after the reduction processing is performed, and acquires image information on the inside of the object.

A workstation or the like is normally used for the signal processor 19. Reduction processing to reduce the acoustic signal generated on the surface of the object, image reconstruction processing and the like are performed by the signal processor 19 according to pre-programmed software. For example, software used for a workstation is constituted by two modules: a signal processing module 19a and an image reconstruction module 19b. The signal processing module 19a performs the reduction processing to reduce the acoustic signal generated on the surface of the object, and noise reduction processing, which are characteristics of this embodiment. The image reconstruction module 19b reconstructs an image using the signals processed by the signal processing module 19a. In photoacoustic tomography, which is one type of photoacoustic imaging, noise reduction processing and the like are performed for a signal detected at each position as a pre-processing of image reconstruction. It is preferable that these processing be performed by the signal processing module 19a.

Furthermore, the image reconstruction module 19b generates image information for image reconstruction. For the image reconstruction algorithm, time domain or Fourier domain back-projection, commonly used in tomographic technology, is used. If sufficient time can be taken for the image reconstruction, then such an image reconstruction method as an inverse problem analysis method, based on repeat processing, is effective. Typical examples of the image reconstruction method used for photoacoustic tomography, which is one type of photoacoustic imaging, are: a Fourier transform method, a universal back-projection method and a filtered back-projection method.

In photoacoustic imaging, an optical characteristic distribution image inside a living body can be generated without reconstructing the image by using a focused acoustic wave detector or by focusing the light. In this case, signal processing using an image reconstruction algorithm is unnecessary.

In some cases, the signal collector 18 and the signal processor 19 may be integrated. In this case, the image information of the object may be generated by hardware processing, instead of the software processing executed by a workstation.

Furthermore, in some cases the signal processor 19 may include a function to control the entire image capturing flow shown in FIG. 3. In this case, the signal processor 19 is electrically connected to the light source 11 and the light source unit 22, and functions as a light source controller that controls wavelength and light quantity. The signal processor 19 could function as a system controller as well.

The object information acquiring apparatus may include an imaging function to automatically perform processing according to the processing flow in FIG. 3 when imaging begins, and provide image information where the signals of hemoglobin have been erased, or image information where only the signals of the contrast agent have been extracted.

(Holding Member)

To stabilize the shape of the object and increase the accuracy of the photoacoustic wave detection and image reconstruction, an object holding member (not illustrated) may be installed. For example, two plate-shaped members to hold the object may be used as the holding member. Another example of the holding member is a cup-shaped, plate-shaped or bowl-shaped member that holds a suspended breast or the like. The holding member preferably has transparency with respect to light and acoustic waves. For example, acrylic or PET resin can be used for the holding member.

(Acoustic Matching Material)

It is preferable to dispose an acoustic matching material between the object and the acoustic wave detector to match the respective acoustic impedances. If the holding member is installed, the acoustic matching material is disposed between the holding member and the object, and between the holding member and the acoustic wave detector. For the acoustic matching material, water, castor oil and ultrasonographic gel, for example, can be suitably used.

(Configuration for Control Information)

It is preferable that the object information acquiring apparatus acquires the control information of the apparatus, and uses this information to acquire a high definition image. The control information is normally the irradiation light quantity at each wavelength. For example, light having each wavelength is measured by a later mentioned light quantity meter, and the light quantity is adjusted to match the optimum control value predetermined according to the depth in the object and the optical characteristics of the object. The user may input the control information using such an input unit as a mouse and keyboard, with reference to the detection signal or reconstructed image at each wavelength, or with reference to a difference detection signal or difference reconstructed image acquired after the subtraction processing. As a function of the input unit, removing or not removing a signal of hemoglobin from the detection signal or reconstructed image may be selectable. If the contrast agent is used, setting or not setting the contrast agent enhancement mode may be selectable.

(Display Device 20)

The display device 20 displays image information that is output from the signal processor 19. For the display device, a liquid crystal display, a plasma display, an organic EL display or the like can be used. The display device may be integrated with the object information acquiring apparatus of this embodiment, or may be a standalone device.

(Light Quantity Meter 21)

The light quantity meter is an apparatus to measure the quantity of the light output from the light source. The light quantity can be measured by, for example, detecting light 121 which is branched light of the light 12 output from the light source. The measured data is transmitted to the signal processor which is electrically connected to the light quantity meter. For the light quantity meter, various conventional types of meters, such as an optical element type, semiconductor type and chemical type, can be used.

As described above, according to this embodiment, the influence of components other than the measurement target can be reduced in the information acquired by PAI. In other words, signals originating in a substance other than the measurement target (e.g. hemoglobin in the case of administering a contrast agent) in the object can be accurately reduced with a minimal number of measurement times (or with a short measurement time). As a result, the influence of hemoglobin or the like on the electric signals and characteristic information can be reduced, and signals of the contrast agent can be acquired well. Furthermore, the burden on the testee can be decreased.

### OTHER EMBODIMENTS

The present invention can also be carried out as a computer (or such a device as a CPU and MPU) of a system or an apparatus that implements the above mentioned functions of the embodiment by reading and executing a program recorded in a storage device. Further, the present invention can also be carried out as a method constituted as steps executed by a computer of a system or an apparatus that implements the above mentioned functions of the embodiment by reading and executing a program recorded in a storage device. For this purpose, the program is provided to the computer via a network or by various types of recording media that functions as the above mentioned storage device (that is, a computer-readable recording media that holds data non-temporarily). Therefore the above mentioned computer (including such a device as a CPU and MPU), the above mentioned method, the above mentioned program (including program codes and program products), and a computer-readable recording media which holds data non-temporarily, are all included within the scope of the present invention.

According to the present invention, the influence of components other than the measurement target in the information acquired by PAI can be reduced.

### OTHER EMBODIMENTS

Embodiments of the present invention can also be realized by a computer of a system or apparatus that reads out and executes computer executable instructions recorded on a storage medium (e.g., non-transitory computer-readable storage medium) to perform the functions of one or more of the above-described embodiment(s) of the present invention, and by a method performed by the computer of the system or apparatus by, for example, reading out and executing the computer executable instructions from the storage medium to perform the functions of one or more of the above-described embodiment(s). The computer may comprise one or more of a central processing unit (CPU), micro processing unit (MPU), or other circuitry, and may include a network of separate computers or separate computer processors. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-144047, filed on Jul. 21, 2015, and, Japanese Patent Application No. 2016-107261, filed on May 30, 2016, which are hereby incorporated by reference herein in their entirety.

1. An object information acquiring apparatus, comprising:
a light source configured to radiate first light having a first wavelength λ1 and second light having a second wavelength λ2;
a detection unit configured to detect an acoustic wave, which is generated from an object irradiated with the light from the light source, and convert the acoustic wave into a detection signal;
a signal processing unit configured to acquire characteristic information of an inside of the object based on the detection signal; and
a light intensity acquiring unit configured to acquire incident light intensity of the light that is radiated from the light source into the object, wherein
the signal processing unit acquires the characteristic information by performing subtraction processing between a signal generated as a result of the first light being absorbed by hemoglobin and a signal generated as a result of the second light being absorbed by hemoglobin,
the first wavelength is 780 nm or more and 810 nm or less, and the second wavelength is 840 nm or more and 920 nm or less, and
an adjustment is performed so that Φ(λ1)≤Φ(λ2) is satisfied, and a difference between the Φ(λ1) and Φ(λ2) is within a predetermined range, where the Φ(λ1) denotes the incident light intensity of the first light to the object, and the Φ(λ2) denotes the incident light intensity of the second light to the object, which are acquired by the light intensity acquiring unit.

2. The object information acquiring apparatus according to claim 1, wherein
the Φ(λ2) is 1.1 times or more and 1.8 times or less than the Φ(λ1).

3. The object information acquiring apparatus according to claim 1, wherein
the signal generated as a result of the first light being absorbed by hemoglobin is a first detection signal which the detection unit has converted from the acoustic wave generated from the object irradiated with the first light, and the signal generated as a result of the second light being absorbed by hemoglobin is a second detection signal which the detection unit has converted from the acoustic wave generated from the object irradiated with the second light.

4. The object information acquiring apparatus according to claim 1, wherein
the signal processing unit generates a reconstructed image based on the detection signal, and
the signal generated as a result of the first light being absorbed by hemoglobin is a reconstructed image originating in the first detection signal which the detection unit has converted from the acoustic wave generated from the object irradiated with the first light, and the signal generated as a result of the second light being absorbed by hemoglobin is a reconstructed image originating in the second detection signal which the detection unit has converted from the acoustic wave generated from the object irradiated with the second light.

5. The object information acquiring apparatus according to claim 1, wherein
the light source radiates the first light and the second light, so that a signal S_{H}(λ1), which is generated as a result of the first light being absorbed by hemoglobin, and a signal S_{H}(λ2), which is generated as a result of the second light being absorbed by hemoglobin, satisfy S_{H}(λ1)≤S_{H}(2).

6. The object information acquiring apparatus according to claim 1, wherein
the light source radiates the first light and the second light so as to satisfy
μ_{HbO2}(λ1)×Φ(λ1,d)≤μ_{HbO2}(λ2)×Φ(λ2,d) and μ_{Hb}(λ1)×Φ(λ1,d)≤μ_{Hb}(λ2)×Φ(λ2,d),
where μ_{HbO2}(λ1) denotes a light absorption coefficient of oxyhemoglobin of the first light, μ_{HbO2}(λ2) denotes a light absorption coefficient of oxyhemoglobin of the second light, μ_{Hb}(λ1) denotes a light absorption coefficient of deoxyhemoglobin of the first light, μ_{Hb}(λ2) denotes a light absorption coefficient of deoxyhemoglobin of the second light, Φ(wavelength, d) denotes a light quantity of the light at depth d in the object from which the characteristic information is acquired, and λ1<λ2.

7. The object information acquiring apparatus according to claim 1, wherein
the light source radiates the first light and the second light, so as to satisfy Φ(λ1)≤Φ(λ2).

8. The object information acquiring apparatus according to claim 1, wherein
the light source radiates the first light and the second light, so as to satisfy
Φ(λ1)=Φ(λ2)×(1+2q/100)
when a fluctuation range from a set value of an output of the light source is ±q % (q is a positive value).

9. The object information acquiring apparatus according to claim 1, wherein
a contrast agent has been administered to the object, and
as the characteristic information, the signal processing unit acquires information on distribution of the contrast agent after the subtraction processing being performed.

10. The object information acquiring apparatus according to claim 6, wherein
a contrast agent has been administered to the object, and
the contrast agent that is used satisfies
μ_{CA}(λ1)×Φ(λ1,d)>μ_{CA}(λ2)×Φ(λ2,d),
where μ_{CA}(λ1) denotes a light absorption coefficient of the contrast agent of the first light, μ_{CA}(λ2) denotes a light absorption coefficient of the contrast agent of the second light, and λ1<λ2.

11. The object information acquiring apparatus according to claim 9, wherein
the contrast agent includes conjugate of indocyanine green and polyethylene glycol.

12. The object information acquiring apparatus according to claim 9, wherein
the contrast agent includes conjugate of indocyanine green and human serum albumin.

13. The object information acquiring apparatus according to claim 1, wherein
the first wavelength and the second wavelength are selected from a range 780 nm to 920 nm.

14. An object information acquiring method, comprising:
a step of radiating first light having a first wavelength λ1 and second light having a second wavelength λ2 from a light source;
a step of a detection unit detecting an acoustic wave, which is generated from an object irradiated with light from the light source, and converting the acoustic wave into a detection signal;
a step of a signal processing unit acquiring characteristic information of an inside of the object based on the detection signal;
a step of acquiring incident light intensity of the light that is radiated from the light source into the object; and
a step of performing an adjustment so that Φ(λ1)≤Φ(λ2) is satisfied, and a difference between the Φ(λ1) and Φ(λ2) is within a predetermined range, where Φ(λ1) denotes the incident light intensity of the first light to the object, and Φ(λ2) denotes the incident light intensity of the second light, wherein
the first wavelength is 780 nm or more and 810 nm or less, and the second wavelength is 840 nm or more and 920 nm or less, and
in the step of acquiring the characteristic information, the signal processing unit acquires the characteristic information by performing subtraction processing between a signal generated as a result of the first light being absorbed by hemoglobin and a signal generated as a result of the second light being absorbed by hemoglobin.
